(19) United States
(12) Patent Application Publication
(10) Pub. No.: US 2012/0143055 A1
(43) Pub. Date: Jun. 7, 2012

(54) METHOD AND SYSTEM FOR ULTRASOUND IMAGING

(75) Inventors: Gary Cheng How Ng, Bothell, WA (US); Jennifer Martin, North Prairie, WI (US)

(73) Assignee: GENERAL ELECTRIC COMPANY, Schenectady, NY (US)

(21) Appl. No.: 12/957,796

(22) Filed: Dec. 1, 2010

(51) Int. Cl. A61B 8/14

(52) U.S. Cl. 600/439

(57) ABSTRACT

A method and system for ultrasound imaging includes tracking the position and orientation of an ultrasound probe. The method and system includes tracking the position and orientation of an instrument while moving the instrument. The method and system includes acquiring ultrasound data of a plane defined along a longitudinal axis of the instrument, where the position of the plane is determined based on the position and orientation of the ultrasound probe and the position and orientation of the instrument. The method and system includes generating a plurality of images of the plane based on the ultrasound data and displaying the plurality of images of the plane as part of a dynamic image.

### FIELD OF THE INVENTION

This disclosure relates generally to a method and system for displaying an image of a plane defined along a longitudinal axis of an instrument.

### BACKGROUND OF THE INVENTION

A conventional ultrasound imaging system comprises an array of ultrasonic transducer elements for transmitting an ultrasound beam and receiving a reflected beam from an object being studied. By selecting the time delay (or phase) and amplitude of the applied voltages, the individual transducer elements can be controlled to produce ultrasonic waves which combine to form a net ultrasonic wave that travels along a preferred vector direction and is focused at a selected point along the beam. Conventional ultrasound imaging systems may also use other focusing strategies. For example, the ultrasound imaging system may control the transducer elements to emit a plane wave. Multiple firings may be used to acquire data representing the same anatomical information. The beamforming parameters of each of the firings may be varied to provide a change in maximum focus or otherwise change the content of the received data for each firing, e.g., by transmitting successive beams with the focal point of each beam being shifted relative to the focal point of the previous beam. By changing the time delay (or phase) of the applied pulses, the beam with its focal point can be moved to scan the object.

The same principles apply when the transducer array is employed to receive the reflected sound energy. The voltages produced at the receiving elements are summed so that the net signal is indicative of the ultrasound reflected from a single focal point in the object. As with the transmission mode, this focused reception of the ultrasonic energy is achieved by imparting a separate delay and gain to the signal from each receiving element. For receive beam-forming, this is done in a dynamic manner in order to focus appropriately for the depth range in question.

Conventional ultrasound systems may be used to help guide an instrument, such as a biopsy needle, within a patient's body. According to one type of conventional system, a needle guide may be mounted to an ultrasound probe in a fixed orientation. The fixed orientation allows for the ultrasound probe to acquire ultrasound data of a region or volume including the needle. The operator may then use the image in order to guide the needle to the desired anatomical region. However, there are several limitations to this conventional technique. First and most significantly, since the ultrasound probe and the needle guide are in a fixed orientation, the operator is not given the flexibility to optimize both the image or the needle guide placement. For example, there may be ultrasound opaque materials, such as bone, obstructing the target structure of the patient. These ultrasound opaque materials may make it difficult or impossible to both obtain a clear image of the target structure and position the ultrasound probe/needle guide in a position to safely obtain a biopsy of the target region.

According to another type of conventional system, the position of the needle guide and or the ultrasound probe may be tracked with a tracking device such as an electromagnetic sensor. The conventional systems typically register the real-time positions of the needle guide and ultrasound probe to previously acquired three-dimensional, hereinafter 3D, image data. For example, the real-time positions of the needle guide and ultrasound probe may be registered to a CT image. Then, using software, the conventional system may project a vector showing the path of the biopsy needle on the previously acquired 3D image. While this technique allows the operator to position the needle guide independently of the ultrasound probe, problems can occur since the operator is relying on previously acquired data to position the needle guide. For example, the patient may be positioned in a different manner and/or the patient's anatomy may have changed its relative orientation since the 3D image was acquired.

For these and other reasons an improved ultrasound imaging system and method for guiding an instrument, such as a needle guide, is desired.

### BRIEF DESCRIPTION OF THE INVENTION

The above-mentioned shortcomings, disadvantages and problems are addressed herein which will be understood by reading and understanding the following specification.

In an embodiment, an ultrasound imaging system includes an ultrasound probe, a first sensor attached to the ultrasound probe, a second sensor attached to an instrument, a display device and a processor in electronic communication with the ultrasound probe, the first sensor, and the second sensor. The processor being configured to receive first data from the first sensor, the first data including position and orientation information for the ultrasound probe. The processor being configured to receive second data from the second sensor, the second data including position and orientation information for the instrument. The processor being configured to control the ultrasound probe to acquire ultrasound data, the ultrasound data including data of a plane defined along a longitudinal axis of the instrument. The processor being configured to use the first data and the second data when acquiring the ultrasound data. The processor being configured to generate an image of the plane based on the ultrasound data and display the image of the plane on the display device.

In another embodiment, a method of ultrasound imaging includes acquiring first data, the first data including position and orientation information for an ultrasound probe. The method includes acquiring second data, the second data including position and orientation information for an instrument. The method includes using the first data and the second data to acquire ultrasound data with the ultrasound probe, the ultrasound data including data of a plane defined along a longitudinal axis of the instrument. The method includes generating an image of the plane based on the ultrasound data. The method includes displaying the image. The method also includes using the image to position the instrument.

In another embodiment, a method of ultrasound imaging includes tracking the position and orientation of an ultrasound probe. The method includes tracking the position and orientation of an instrument while moving the instrument. The method includes acquiring ultrasound data of a plane defined along a longitudinal axis of the instrument, where the position of the plane is determined based on the position and orientation of the ultrasound probe and the position and orientation of the instrument. The method includes generating a plurality of images of the plane based on the ultrasound data and displaying the plurality of images of the plane as part of a dynamic image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an ultrasound imaging system in accordance with an embodiment;

FIG. 2 is a schematic representation of an ultrasound imaging system in accordance with an embodiment;

FIG. 3 is a schematic representation of a biopsy needle and a sensor assembly in a partially exploded view in accordance with an embodiment;

FIG. 4 is a schematic representation of a biopsy needle and a sensor assembly in a fully assembled view in accordance with an embodiment;

FIG. 5 is a schematic representation of a detailed perspective view of an ultrasound probe and a sensor in accordance with an embodiment;

FIG. 6 is a flow chart of a method in accordance with an embodiment; and

FIG. 7 is a schematic representation of a plane that is defined along a longitudinal axis of an instrument.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

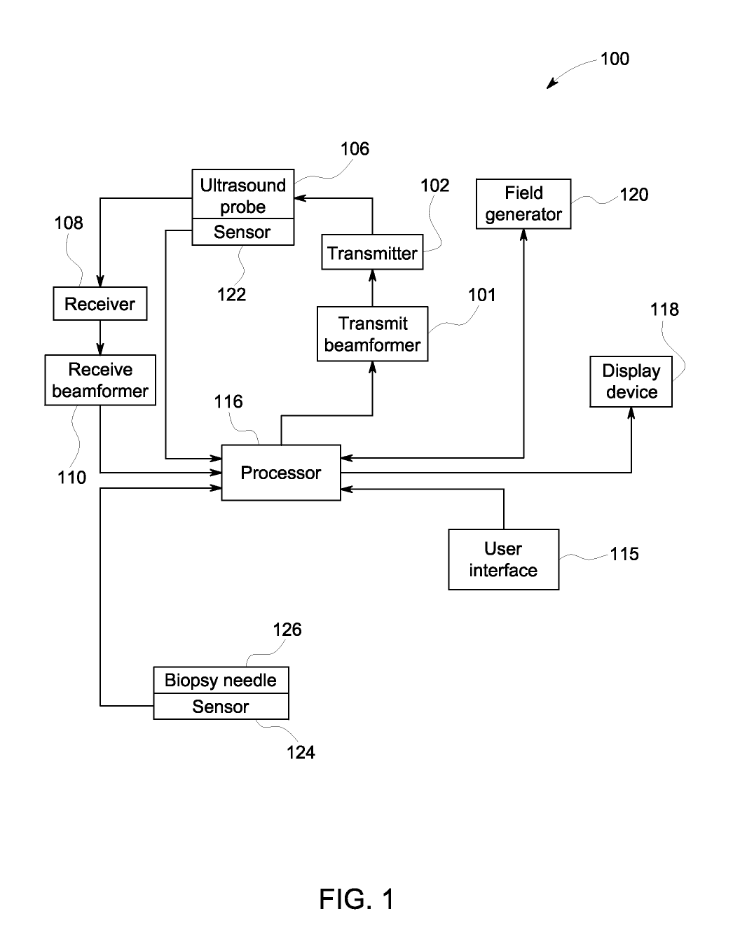

FIG. 1 is a schematic diagram of an ultrasound imaging system 100 in accordance with an embodiment. The ultrasound imaging system 100 includes a transmit beamformer 101 and a transmitter 102 that drive transducer elements (not shown) within an ultrasound probe 106 to emit pulsed ultrasonic signals into a body (not shown). A variety of geometries of ultrasound probes and transducer elements may be used. The pulsed ultrasonic signals are back-scattered from structures in the body, like blood cells or muscular tissue, to produce echoes that return to the transducer elements. The echoes are converted into electrical signals, or ultrasound data, by the transducer elements in the ultrasound probe 106 and the electrical signals are received by a receiver 108. According to other embodiments, the ultrasound probe 106 may contain electronic circuitry to do all or part of the transmit and/or the receive beam forming. For example, all or part of the transmit beamformer 101, the transmitter 102, the receiver 108 and the receive beamformer 110 may be disposed within the ultrasound probe 106 according to other embodiments. The terms “scan” or “scanning” may also be used in this disclosure to refer to acquiring ultrasound data through the process of transmitting and receiving ultrasonic signals. For purposes of this disclosure, the term “ultrasound data” may include data that was acquired and/or processed by an ultrasound system. Additionally, the term “data” may also be used in this disclosure to refer to either one or more datasets. The electrical signals representing the received echoes are passed through the receive beam-former 110 that outputs ultrasound data. A user interface 115 may be used to control operation of the ultrasound imaging system 100, including, to control the input of patient data, to change a scanning or display parameter, and the like.

The ultrasound imaging system 100 also includes a processor 116 in electronic communication with the ultrasound probe 106. The processor 116 may control the transmit beamformer 101 and the transmitter 102, and therefore, the ultrasound signals emitted by the transducer elements in the ultrasound probe 106. The processor 116 may also process the ultrasound data into images for display on a display device 118. According to an embodiment, the processor 116 may also include a complex demodulator (not shown) that demodulates the RF ultrasound data and generates raw ultrasound data. The processor 116 may be adapted to perform one or more processing operations according to a plurality of selectable ultrasound modalities on the ultrasound data. The ultrasound data may be processed in real-time during a scanning session as the echo signals are received. For the purposes of this disclosure, the term “real-time” is defined to include a procedure that is performed without any intentional delay. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors to handle the processing tasks described hereinabove.

The ultrasound imaging system 100 may continuously acquire ultrasound data at a frame rate of, for example, 10 Hz to 30 Hz. Images generated from the ultrasound data may be refreshed at a similar frame rate. Other embodiments may acquire and display ultrasound data at different rates. For example, some embodiments may acquire ultrasound data at a frame rate of less than 10 Hz or greater than 30 Hz depending on the size of the region or volume being scanned and the intended application. A memory (not shown) may be included for storing processed frames of acquired ultrasound data. In an embodiment, the memory may be of sufficient capacity to store at least several seconds worth of frames of ultrasound data. The frames of ultrasound data are stored in a manner to facilitate retrieval thereof according to its order or time of acquisition. The memory may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, ultrasound data may be processed by different mode-related modules (e.g., B-mode, Color Doppler, M-mode, Color M-mode, spectral Doppler, TVI, strain, strain rate, and the like) to form 2D or 3D data sets of image frames and the like. For example, one or more modules may generate B-mode, color Doppler, M-mode, color M-mode, spectral Doppler, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

The ultrasound imaging system 100 also includes a field generator 120 according to an embodiment. The field generator 120 may comprise one or more sets of coils adapted to pass an electric current in order to generate an electromagnetic field. The ultrasound imaging system 100 also includes a first sensor 122 attached to the ultrasound probe 106 and a second sensor 124 attached to a biopsy needle 126. The second sensor 124 may be attached to instruments other than a biopsy needle according to other embodiments. The processor 116 is in electronic communication with the first sensor 122 and the second sensor 124. The first sensor 122 and the second sensor 124 may each comprise an electromagnetic sensor. According to an embodiment, the first sensor 122 and the second sensor 124 each include three sets of coils disposed orthogonally to each other. For example, a first set of coils may be disposed along an x-axis, a second set may be disposed along a y-axis, and a third set may be disposed along a z-axis. Different currents are induced in each of the three orthogonal coils by the electromagnetic field from the field generator 120. By detecting the currents induced in each of the coils, position and orientation information may be determined for both the first sensor 122 and the second sensor 124. According to the embodiment shown in the imaging system 100, the first sensor 122 is attached to the ultrasound probe 106. The processor 116 is able to determine the position and orientation of the ultrasound probe 106 based on the data from the first sensor 122. Likewise, the processor 116 is thus able to determine the position and orientation of the biopsy needle 126 based on the data received from the second sensor 124. Using a field generator and an electromagnetic sensor to track the position and orientation of an electromagnetic sensor within an electromagnetic field is well-known by those skilled in the art and, therefore, will not be described in additional detail. While the embodiment of FIG. 1 uses a field generator and electromagnetic sensors, it should be appreciated by those skilled in the art that other embodiments may use other methods of obtaining position and orientation information for an ultrasound probe and an instrument. For example, embodiments may use optical tracking systems, including systems where multiple light-emitting diodes (LEDs) or reflectors are attached to both an ultrasound probe and an instrument, and a system of cameras is used to determine the position of the LEDs or reflectors through triangulation or other methods.

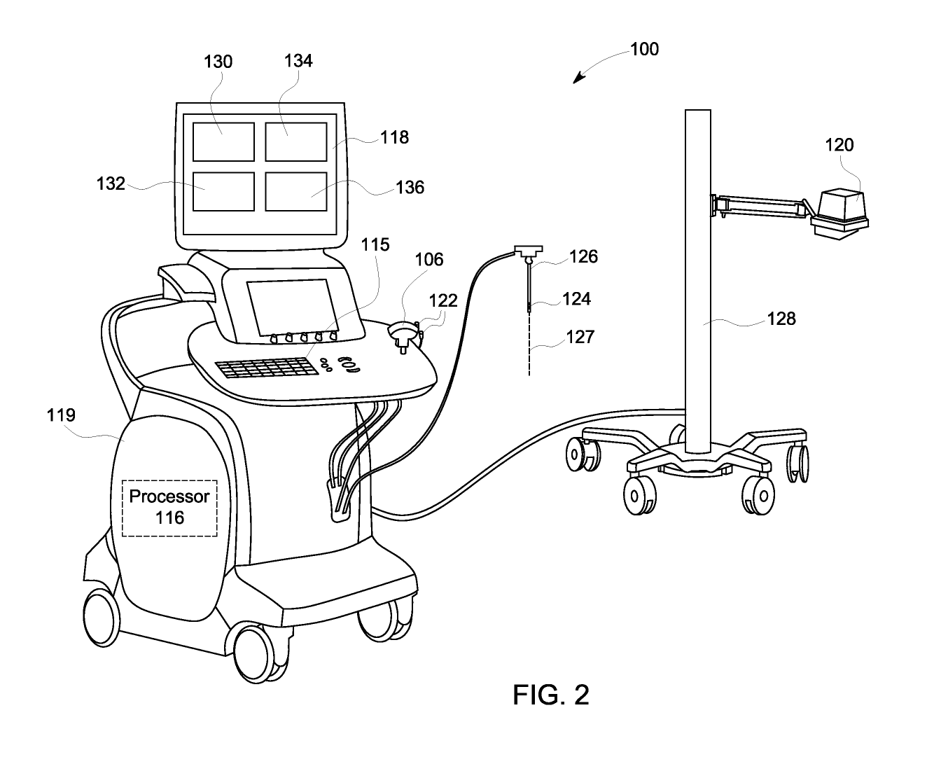

FIG. 2 is a schematic representation of the ultrasound imaging system 100 from FIG. 1 in accordance with an embodiment. For simplicity, common reference number will be used to identify identical components within FIGS. 1 and 2. Additionally, components that were previously described with respect to FIG. 1 may not be described in detail with respect to FIG. 2.

Referring to FIG. 2, the processor 116 is disposed in a cart-style ultrasound imaging system 119. The first sensor 122 is attached to the ultrasound probe 106. The second sensor 124 is attached to the biopsy needle 126. A longitudinal axis 127 of the biopsy needle 126 is represented with a dashed line. According to an embodiment, the longitudinal axis 127 may be oriented along the biopsy needle 126. In other words, the longitudinal axis 127 may indicate the insertion path of the biopsy needle 126 from a given orientation. The ultrasound probe 106 may comprise an ultrasound probe capable of acquiring three-dimensional ultrasound data. The ultrasound probe 106 may be able to acquire ultrasound data of a plane of any position and orientation within a possible acquisition volume. The ultrasound probe 106 shown in FIG. 2 is a matrix type three-dimensional ultrasound probe with an array of elements that are fully steerable in both the elevation and azimuth directions. Other embodiments may use other types of ultrasound probes such as a mechanical swept ultrasound probe with one or more rows of elements that are swept through an arc in order collect ultrasound data along different vectors.

The display device 118 may be a flat panel LCD screen. FIG. 2 shows the display device 118 divided into four section in accordance with an embodiment: a first section 130, a second section 132, a third section 134, and a fourth section 136. The size, orientation and number of sections shown on the display 118 may be user configurable. Other embodiments may use a display device that is not divided into sections like the display device 118. For example, other embodiments may use a display device divided into either a different number of sections and/or the sections may be configured in a different manner. Additional information about the types of images shown on the four sections of the display device 118 in accordance with an embodiment will be described in detail hereinafter. The field generator 120 is shown affixed to a cart 128 according to an embodiment.

FIG. 3 is a schematic representation of the biopsy needle 126 of FIGS. 1 and 2 and a sensor assembly 156 in a partially exploded view in accordance with an embodiment.

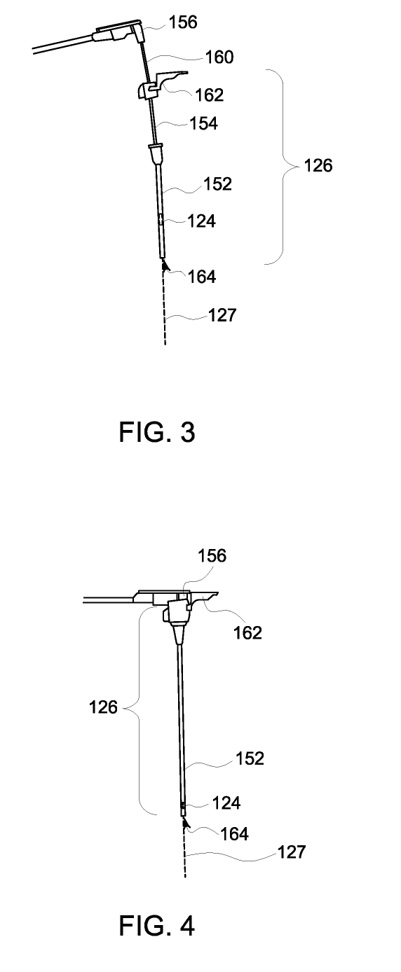

FIG. 4 is a schematic representation of the biopsy needle 126 and the sensor assembly 156 of FIG. 3 in a fully assembled view in accordance with an embodiment.

Referring to both FIG. 3 and FIG. 4, the biopsy needle 126 includes a sheath 152 and a stylet 154. The sheath may be a 16 gauge tube. The stylet 154 may be an 18 gauge tube sized to fit within the inner diameter of the sheath 152. The sensor assembly 156 includes the second sensor 124 connected to a sensor extender 160. The second sensor 124 may include three or more coils disposed at orthogonal angles to each other. The sensor extender 160 may include three or more wires carrying signals from the electromagnetic sensor 156. The biopsy needle 126 also includes a latch 162 adapted to secure the stylet 154 inside the sheath 152. The latch 162 is also adapted to engage the sensor assembly 156. The longitudinal axis 127 of the biopsy needle 126 is also schematically represented by a dashed line. The sheath 152 and stylet 154 of the biopsy needle 126 are both generally tubular structures. The longitudinal axis 127 is defined to include an axis passing through the center of the stylet 154 and the sheath 152 when the biopsy needle 126 is assembled as in FIG. 4. As mentioned previously, a biopsy needle, such as the biopsy needle 126, is just one example of an instrument (shown in FIG. 1) that may be tracked with a sensor. Other embodiments may include an instrument selected from the non-limiting list including a catheter and an ablation electrode. For embodiments using an instrument other than a biopsy needle, the term “longitudinal axis” may be defined to include an axis oriented in the long direction of the instrument and generally centered in the instrument. For instruments that are designed to be inserted into a patient, the term “longitudinal axis” is also defined to include an axis oriented along the path in which the instrument is designed to be inserted into the patient.

According to an embodiment, the second sensor 124 may be positioned at a fixed distance from a distal end 164 of the biopsy needle 126 as shown in the fully-assembled biopsy needle 126 and sensor assembly 156 of FIG. 4. When placed in an electromagnetic field, the second sensor 124 is adapted to rely data about the position and orientation of the second sensor 124 through the sensor extender 160 and to the processor 116 (shown in FIG. 1). When the biopsy needle and the sensor assembly 156 are fully-assembled as in FIG. 4, the second sensor 124 is in a known position with respect to stylet 154 and the sheath 152. Therefore, the data from the electromagnetic sensor 124 may also be used to determine the position and orientation of the stylet 154 and the sheath 152. The processor 116 may track the position and orientation of an instrument, in this case the biopsy needle 126, by calculating the position and orientation of the of the second sensor 124 at a plurality of different sample times.

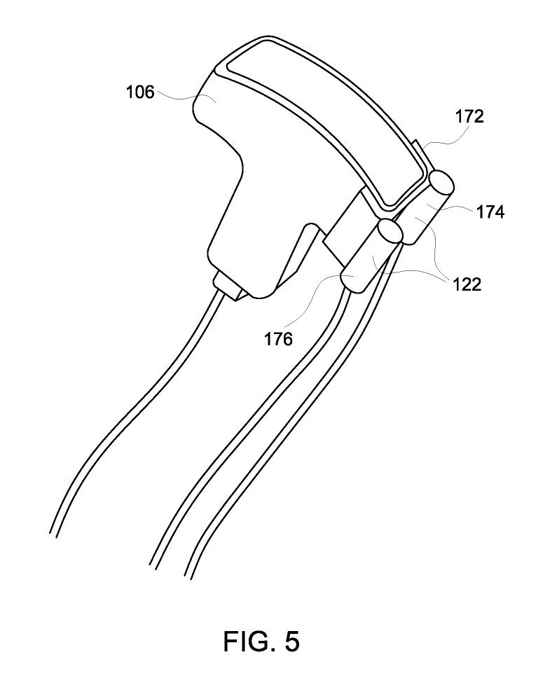

FIG. 5 is a schematic representation of a detailed perspective view of the ultrasound probe 106 and the first sensor 122 from the ultrasound imaging system 100 of FIG. 2 in accordance with an embodiment. The first sensor 122 may be attached to the ultrasound probe 106 by a bracket 172 that allows for the first sensor 122 to be easily attached or removed to the ultrasound probe 106. The first sensor 122 comprises a first electromagnetic sensor portion 174 and a second electromagnetic sensor portion 176 according to an embodiment. Signals from the first electromagnetic sensor portion 174 and the second electromagnetic sensor portion 176 may be used to determine the position and orientation of the ultrasound probe 106 when placed in a known electromagnetic field. The processor 116 (shown in FIG. 1) may track the position and orientation of the ultrasound probe 106 by calculating the position and orientation of the first sensor 122 multiple times over a period of time.

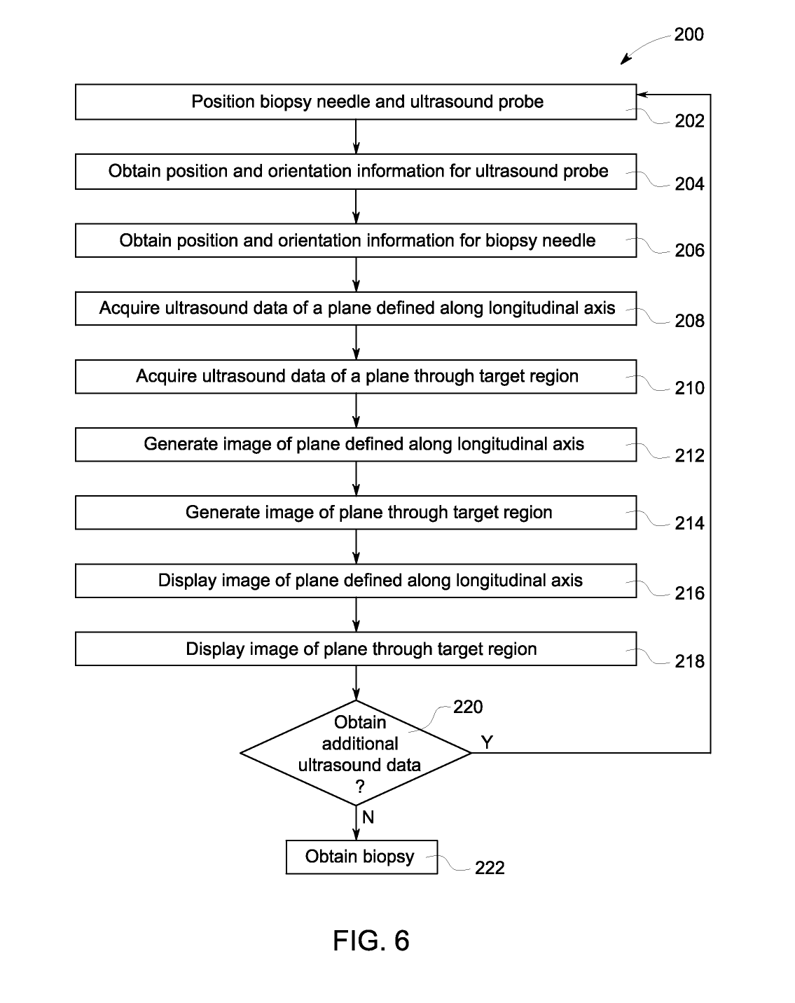

FIG. 6 is a flow chart of a method in accordance with an embodiment. The individual blocks represent steps that may be performed in accordance with the method 200. Additional embodiments may perform the steps shown in a different sequence and/or additional embodiments may include additional steps not shown in FIG. 2. The technical effect of the method 200 is the display of an image of a plane defined along a longitudinal axis of a biopsy needle and the display of a second image of a second plane through a target region.

According to an exemplary embodiment, the method 200 may be performed with an ultrasound imaging system such as the ultrasound imaging system 100 shown in FIG. 2. Referring to both FIG. 2 and FIG. 6, at step 202 a user positions the biopsy needle 126 and the ultrasound probe 106. Since the user is attempting to obtain a biopsy of the patient, the user may position the ultrasound probe 106 in a position to show a target region from which the biopsy is desired. Additionally, the user may start by positioning the biopsy needle 126 at his/her best guess for a location from which to obtain the biopsy from the target region. If the user is actively scanning the patient with the ultrasound probe 106 while positioning the biopsy needle 126, then the user may use a real-time dynamic ultrasound image to help initially position the biopsy needle 126.

At step 204, the processor 116 obtains first data indicating the position and orientation of the ultrasound probe 106. At step 206, the processor 116 obtains second data indicating the position and orientation of the biopsy needle 126. As described hereinabove, the first sensor 122 is attached to the ultrasound probe 106 and the second sensor 124 is attached to the biopsy needle. The processor 116 may calculate the position and orientation of both the ultrasound probe 106 and the biopsy needle 126 in an electromagnetic field of a known strength and orientation that is emitted from the field generator 120 as was described previously. The processor 116 is also able to calculate the relative position of the ultrasound probe 106 with respect to the biopsy needle 126 by comparing the signals received from the first sensor 122 to the signals received from the second sensor 124.

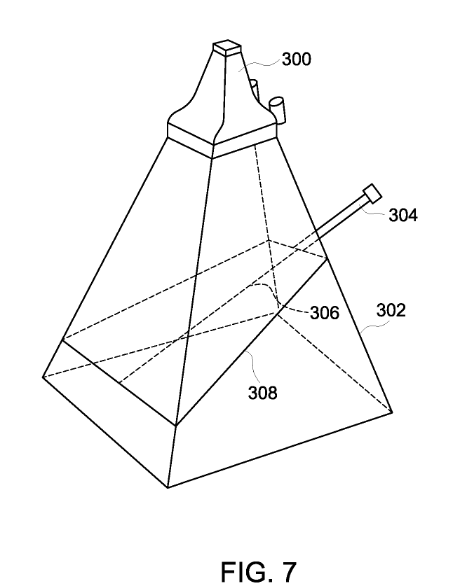

At step 208, the processor 116 controls the ultrasound probe 106 to acquire ultrasound data of a plane defined along the longitudinal axis 127 of the biopsy needle 126. The processor 116 utilizes the data acquired from the first sensor 122 and the second sensor 124 in order to determine the position of the plane defined along the longitudinal axis 127 in relation to the ultrasound probe 106. An example of a plane defined along a longitudinal axis of an instrument, such as a biopsy needle, will be discussed hereinafter with respect to FIG. 7.

At step 210, the processor 116 controls the ultrasound probe 106 to acquire second ultrasound data. According to an embodiment, the second ultrasound data includes data of a second plane through a target region. The target region may, for instance, be identified prior to the start of the method 200. For example, according to an embodiment, the user may indicate the location of the target region on an image acquired with the ultrasound probe 106. The processor 116 is then able to correlate the information about the indicated target region on the screen with the first data from the first sensor 122 indicating the position and orientation of the ultrasound probe 106 while the image was acquired. According to an embodiment, the user may identify the target region before the start of method 200.

Thus, according to an embodiment, the processor 116 may use a priori information regarding the location of the target region. The processor 116 may then use feedback regarding the real-time position and orientation of the ultrasound probe 106 in order to control the transducer elements in the ultrasound probe 106 to acquire second ultrasound data of a second plane through the target region during step 210. According to an embodiment, the second plane, which passes through the target region, may be disposed at an angle with respect to the plane defined along the longitudinal axis 127 of the biopsy needle 126. The processor 116 may then generate an image of the plane defined along the longitudinal axis 127 of the biopsy needle 126 at step 212 based on the ultrasound data that was acquired at step 208. At step 214, the processor 116 generates an image of the second plane through the target region based on the data acquired as step 210. At step 216, the processor 116 displays an image of the plane defined along the longitudinal axis 127 of the biopsy needle 126 on the display device 118. Then, at step 218, the processor 116 displays the image of the second plane through the target region on a display device 118.

At step 220, the processor 116 determines if the acquisition of additional ultrasound data is desired. According to an embodiment, if the user continues to scan a patient, the processor 116 may determine that additional ultrasound data is desired. If additional ultrasound data is desired at step 220, the method 200 proceeds to step 202, where steps 202, 204, 206, 208, 210, 212, 214, 216, 218, and 220 are implemented an additional time in accordance with an embodiment. Those skilled in the art should appreciate that the ultrasound data acquired at steps 208 and 210 will be reflective of a later period of time during each successive iteration through steps 202, 204, 206, 208, 210, 212, 214, 216, 218, and 220. According to an embodiment, the image of the plane defined along the longitudinal axis of the biopsy needle may be replaced with an updated image of the plane defined along the longitudinal axis of the biopsy needle at step 216 during each successive iteration of steps 202, 204, 206, 208, 210, 212, 214, 216, 218, and 220. Likewise, the image of the second plane through the target region may be replaced with an updated image of the second plane through the target region at step 218 during each successive iteration of steps 202, 204, 206, 208, 210, 212, 214, 216, 218, and 220. According to an embodiment where the method 200 loops through steps 202, 204, 206, 208, 210, 212, 214, 216, 218, and 220 multiple times, the result may be the generation and display of a dynamic image of a plane defined along the longitudinal axis of the biopsy needle and the generation and display of a dynamic image of a plane through the target region. For purposes of this disclosure, the term “dynamic image” is defined to include a loop comprising multiple images or frames that are acquired at different points in time. When displayed, a dynamic image may be useful because it shows how a region changes over time.

A dynamic image of the plane defined along the longitudinal axis of the biopsy needle may be useful since it shows a view of the intended trajectory of the biopsy needle 126. As such, a user may use this view to correctly position the biopsy needle 126 or other instrument. For example, if an image of the plane defined along the longitudinal axis shows that the biopsy needle 126 would be likely to intersect one or more vital regions of a patient's anatomy, the user may wish to reposition the biopsy needle 126 before puncturing the patient. Additionally, the user may use the dynamic image showing the second plane through the target region in order to help position the biopsy needle 126 so that the user is able to obtain the desired tissue sample. According to an embodiment, an indicator, such as a line, may be shown on the image of the plane defined along the longitudinal axis 127 of the biopsy needle 126. The indicator may show the real-time trajectory of the needle in order to help the operator position the biopsy needle. Likewise, according to an embodiment, a second indicator, such as a highlighted region, may be shown on the image of the second plane through the target region showing the place where the biopsy needle, or other instrument, would intersect the second plane. By acquiring data from just two planes, i.e. a plane defined along the longitudinal axis and the second plane through the target region, it is possible to generate dynamic ultrasound images with either better resolution and/or faster refresh rates than methods where a larger volume of ultrasound data is being acquired for each image. Higher resolution and/or higher frame rates allow the user to quickly and accurately manipulate an instrument into a satisfactory position. According to an embodiment, the refresh rates for the dynamic images may be fast enough to allow for the user to obtain real-time feedback from the dynamic images about the current position of the biopsy needle prior to puncturing the patient. It may be advantageous for the operator to obtain real-time feedback when positioning the biopsy needle because the real-time feedback allows the user to quickly and accurately position the biopsy needle in a location that facilitates the desired tissue biopsy without potentially damaging any surrounding sensitive tissue.

Referring to FIG. 2 and the method 200, according to an embodiment, the dynamic image of the first plane may be displayed in the first section 130 of the display device 118 and the dynamic image of the second plane may be shown in the second section 132. According to embodiments where ultrasound data of additional planes are acquired, either static or dynamic images may be shown in the third section 134 or the fourth section 136 of the display device 118. It should be appreciated that FIG. 2 shows just one exemplary way that the display device 118 may be divided into sections.

Referring to FIG. 6, at step 220, if it is determined that no additional ultrasound data is desired, the method 200 advances to step 222 where a user implements the biopsy needle 126 to obtain a biopsy of the target region. According to another embodiments, the user may obtain a biopsy at any point during consecutive iterations of steps 202, 204, 206, 208, 210, 212, 214, 216, 218, and 220.

FIG. 7 is a schematic representation of one example of a plane that is defined along a longitudinal axis of an instrument. An ultrasound probe 300 is shown along with the potential acquisition volume 302. According to the embodiment shown in FIG. 7, the potential acquisition volume 302 comprises four roughly trapezoidal sides and a bottom side that is rectangular in shape. An instrument 304 is shown outside the potential acquisition volume 302. A longitudinal axis 306 of the instrument 304 is schematically represented by a dashed line. A plane 308 is shown that is defined along the longitudinal axis 306 of the instrument 304. According to an embodiment, the ultrasound probe 300 may be a three-dimensional matrix probe that is capable of being steered in both azimuthal and elevational directions. The ultrasound probe 300 may be controlled to acquire ultrasound data of the plane 308. For example, when used with a method such as the method 200 shown in FIG. 6, ultrasound data of the plane 308 acquired at different points in time may be used to generate and display a dynamic image of the plane. It should be appreciated that plane 308 only shows one possible plane that is defined along the longitudinal axis 306 of the instrument 304.

According to an embodiment where the instrument 304 is a biopsy needle, the ultrasound data of the plane 308 may be used to generate an image showing the potential trajectory of the biopsy needle. As the user manipulates the instrument 304, updated ultrasound datasets of the plane 308 defined along the longitudinal axis of the instrument 304 may be acquired and updated images of the plane 308 may be displayed. Since the plane 308 is defined along the longitudinal axis 306, is should be appreciated that updated ultrasound datasets of the plane 308 may be displayed to show the potential trajectory of the instrument 304 even as the instrument 304 is being manipulated by the user. According to an embodiment, the plane 308 may be defined to have a fixed relationship to the instrument 304, even as the instrument 304 is being manipulated. According to other embodiments, the ultrasound probe 300 may be controlled to acquire a different planes of ultrasound data with respect to the instrument 304 during each successive acquisition. However, according to an embodiment, each of the planes will be defined along the longitudinal axis 306 of the instrument 304 in a manner similar to the plane 308.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasound imaging system comprising:
an ultrasound probe;
a first sensor attached to the ultrasound probe;
a second sensor attached to an instrument;
a display device; and
a processor in electronic communication with the ultrasound probe, the first sensor and the second sensor, the processor configured to:
receive first data from the first sensor, the first data comprising position and orientation information for the ultrasound probe;
receive second data from the second sensor, the second data comprising position and orientation information for the instrument;
control the ultrasound probe to acquire ultrasound data, the ultrasound data comprising data of a plane defined along a longitudinal axis of the instrument, the processor configured to use the first data and the second data when acquiring the ultrasound data;
generate an image of the plane based on the ultrasound data; and
display the image of the plane on the display device.

2. The ultrasound imaging system of claim 1, further comprising a field generator configured to emit an electromagnetic field detectable by the first sensor and the second sensor.

3. The ultrasound imaging system of claim 2, wherein the first sensor is an electromagnetic sensor.

4. The ultrasound imaging system of claim 1, wherein the processor is further configured to use the first data to control the ultrasound probe to acquire second ultrasound data, the second ultrasound data comprising data of a second plane through a target region, the second plane being different than the plane.

5. The ultrasound imaging system of claim 4, wherein the processor is further configured to generate a second image based on the second ultrasound data, the second image comprising an image of the second plane.

6. The ultrasound imaging system of claim 5, wherein the processor is further configured to display the second image on the display device while the image of the plane is being displayed.

7. The ultrasound imaging system of claim 6, wherein the processor is further configured to control the ultrasound probe to acquire third ultrasound data, the third ultrasound data comprising data of a third plane defined along the longitudinal axis of the instrument, the third plane being disposed at an angle with respect to the plane.

8. The ultrasound imaging system of claim 1, wherein the ultrasound probe comprises an ultrasound probe capable of acquiring three-dimensional ultrasound data.

9. The ultrasound imaging system of claim 1, wherein the instrument comprises a biopsy needle.

10. The ultrasound imaging system of claim 1, wherein the instrument comprises a catheter.

11. The ultrasound imaging system of claim 1, wherein the instrument comprises an ablation electrode.

12. A method of ultrasound imaging comprising:
acquiring first data, the first data comprising position and orientation information for an ultrasound probe;
acquiring second data, the second data comprising position and orientation information for an instrument;
using the first data and the second data to acquire ultrasound data with the ultrasound probe, the ultrasound data comprising data of a plane defined along a longitudinal axis of the instrument;
generating an image of the plane based on the ultrasound data;
displaying the image of the plane; and
using the image of the plane to position the instrument.

13. The method of claim 12, further comprising using the first data to acquire second ultrasound data with the ultrasound probe, the second ultrasound data comprising data of a second plane through a target region, the second plane being disposed at an angle with respect to the plane.

14. The method of claim 13, further comprising generating a second image based on the second ultrasound data, the second image comprising an image of the second plane.

15. The method of claim 14, further comprising displaying the second image at generally the same time as the image of the plane.

16. The method of claim 15, further comprising using the second image to position the instrument.

17. The method of claim 12, wherein the image of the plane comprises a frame of a dynamic image.

18. The method of claim 12, wherein the instrument comprises a biopsy needle.

19. A method of ultrasound imaging comprising:
tracking the position and orientation of an ultrasound probe;
tracking the position and orientation of an instrument while moving the instrument;
acquiring ultrasound data of a plane defined along a longitudinal axis of the instrument, where the position of the plane is determined based on the position and orientation of the ultrasound probe and the position and orientation of the instrument;
generating a plurality of images of the plane based on the ultrasound data; and
displaying the plurality of images of the plane as part of a dynamic image.

20. The method of claim 19, where said displaying the plurality of images of the plane as part of a dynamic image occurs in real-time.
